(19) United States
(12) Patent Application Publication
(10) Pub. No.: US 2010/0146799 A1
(43) Pub. Date: Jun. 17, 2010

(54) Guarded Surgical Knife

(75) Inventors: Luther A. Hoffman, Sinking Springs, PA (US); Ronald A. Heim, West Lawn, PA (US); Paul G. Oxholm, Wyomissing, PA (US)

Correspondence Address: MARTIN SAVITZKY;Law Offices of Martin Savitzky Esq., PO BOX 1027, FORT WASHINGTON, PA, 19034-1027, US

(73) Assignee: UNIQUE TECHNOLOGIES INC., Mohnton, PA (US)

(21) Appl. No.: 12/632,764

(22) Filed: Dec. 7, 2009

Related U.S. Application Data
(60) Provisional application No. 61/122,145, filed on Dec. 12, 2008.

(51) Int. Cl. B26B 29/00; A61B 17/32

(52) U.S. Cl. 30/286; 606/167

(57) ABSTRACT

A blade protection device that includes an instrument body, a blade connected to the instrument body, and a guard slidably mounted on, and completely surrounding the instrument body. The guard may be slid between a guard-retracted position in which the blade is exposed for use, and a guard-extended position for covering the sharp cutting edge of the blade. In the guard-retracted position, instrument body may be exposed at both the distal and proximal ends to allow improved control and blade orientation. The retracted guard may allow the user to firmly grip both the proximal and distal portions of the instrument body. The instrument body may be molded as a single piece with the blade holder to prevent unwanted blade or handle movement. The slideably mountable guard may be locked, or latched, in both the guard- extended position and in the guard-retracted position.

### CROSS REFERENCE TO APPLICATIONS

This application is related to, and claims priority from, U.S. Provisional Patent application No. 61/122,145 filed on Dec. 12, 2008, the contents of which are hereby incorporated by reference.

### TECHNICAL FIELD

The present invention relates to safety systems and methods for blades, and more particularly to a safety system and method in which a movable guard may be retracted to expose a blade when the blade is in use, and that can be extended to cover the blade when the blade is not in use.

### BACKGROUND ART

In various surgical procedures, a physician typically has to make an incision in a patient in order to remove unwanted tissue, repair damaged tissue, or implant a device to improve the patient's well being. In certain cases, all three of these activities, or a combination thereof, must be done in a single procedure. Typically a nurse or other surgical assistant manages the devices that are used during such surgeries. For example, the assistant ensures that the appropriate sterile devices are available in the operating suite for the particular procedure that is to be performed. With respect to scalpels, the nurse often hands the scalpel to the physician in a predetermined orientation so that the physician can grip the scalpel's handle without taking his or her eyes away from the patient. This also minimizes the possibility that the physician will be cut with the blade on the scalpel. After the physician completes the incision, the scalpel is handed back to the assistant for later use as part of the procedure or for proper disposal or sterilization. While the procedure is being performed, this requires the assistant to place the used scalpel on a particular tray that will be removed after the procedure is completed. The devices on the tray are then disposed of or are sterilized for reuse. If all appropriate protocols are followed, no hospital personnel should be cut by used or unused scalpel blades. Unfortunately, accidental cuts of hospital personnel do occur for a variety of reasons. Cuts and nicks from blades are uncomfortable and distracting at best. In addition, such cuts and nicks from used blades may result in blood or body fluid exposure that can result in the spread of infectious diseases between the patient and hospital personnel.

These designs typically take the form of a scalpel having a guard that shields the sharp cutting surface of the blade from undesired contact with hospital personnel and surrounding surfaces. The guard in these devices can be extended to a position shielding the blade or retracted to expose the blade for use. Alternatively, the scalpel may be designed to allow the blade to move into or out of the scalpel handle, to either shield or expose the sharp cutting surface. Unfortunately, these designs are deficient because they tend to be cumbersome, difficult to use, expensive to manufacture and may cause unwanted shielding or exposure of the blade prior to the need for such shielding or exposure. Also, such devices may require considerable attention by the user to shield or expose the blade. Additionally, the shield may distort the handle when retracted, making the handle difficult to hold or control. Any design that allows the blade to move, for retraction or extension design purposes, also introduces concerns regarding exact blade positioning and rigidity during use. Such concerns also apply in cases in which the user is required to hold the refracted shield as a grip, allowing any movement between shield and handle to possibly result in unwanted movement of the blade. Accordingly, a need exists for a device and method to provide a shielding mechanism that is simple to use and remains locked in a shielded position until disengaged by the user. The handle and shielding mechanism must also provide a uniform gripping surface when retracted, allowing user control of the scalpel without any unwanted gripping surface or blade movement.

### SUMMARY OF THE INVENTION

### Technical Problem

The technical problem addressed by the present invention includes, but is not limited to, shielding and protecting a sharp blade, such as a scalpel, to minimize the chances of cuts or nicks during handling or disposal of the blade, while allowing the blade to be exposed for use. The technical problem addressed further includes ensuring that the resultant device is easy to use and does not detrimentally alter a users ability to use and control the blade.

### Solution to Problem

The present invention solves the technical problem by providing a blade protection device that includes a handle or instrument body, a blade connected to the instrument body, and a slidably mountable guard that completely surrounds the instrument body. The guard may, by means of a sliding movement, be adjusted between a guard-retracted position in which the blade is exposed for use, and a guard-extended position in which the sharp cutting edge of the blade is covered. In the guard-retracted position, an exposed handle may be provided at both the distal and proximal end of the handle to allow improved handle control and blade orientation. The retracted guard may be positioned to allow the user to firmly grip both the proximal and distal handle portion, which may be molded as a single piece with the blade holder to prevent unwanted blade or handle movement due to guard mechanism tolerances.

The blade protection device may include a means positioned, in part, adjacent to the distal end of the instrument body to lock or latch the guard body in the guard-body-extended position.

The blade protection device may also include a means positioned, in part, adjacent to the proximal end of the instrument body, to provide a means to lock or latch the guard body in the guard-body-retracted position.

### ADVANTAGEOUS EFFECTS OF INVENTION

Advantages effects of the invention include, but are not limited to, providing an easy to use, inexpensive to manufacture, blade shielding device that requires little attention by the user to either shield or expose the blade. The advantages effects of the invention also include improved rigidity and control of blade position during use.

These and other features of the invention will be more fully understood by references to the following drawings.

### BRIEF DESCRIPTION OF DRAWINGS

FIG. 1A shows a 3-D drawing of an exemplary blade protection device latched in a guard-retracted mode.

FIG. 1B shows a 3-D drawing of an exemplary blade protection device latched in a guard-extended mode.

FIG. 2 shows a 3-D drawing of an exemplary slidably mountable guard.

FIG. 3A shows a top elevation of an exemplary instrument body in accordance with the present invention.

FIG. 3B shows a top elevation of an exemplary slidably mountable guard in accordance with the present invention.

FIG. 4A shows an elevation view of an exemplary blade protection device latched in a guard-retracted position.

FIG. 4B shows an elevation view of an exemplary blade protection device unlatched in a guard-retracted position.

FIG. 4C shows an elevation view of an exemplary blade protection device transitioning from a guard-retracted position to a guard-extended position.

FIG. 4D shows an elevation view of an exemplary blade protection device un-latched in a guard-extended position.

FIG. 4E shows an elevation view of an exemplary blade protection device latched in a guard-extended position.

FIG. 5 shows an elevation view of a further exemplary blade protection device latched in a guard-retracted position.

### DESCRIPTION OF EMBODIMENTS

Embodiments of the present invention will now be described in detail by reference to the accompanying drawings in which, as far as possible, like elements are designated by like numbers.

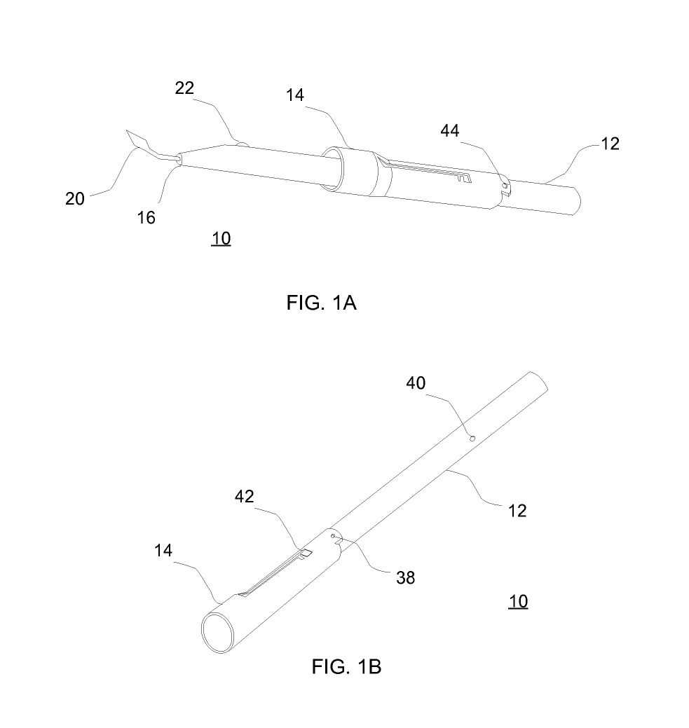

FIG. 1A shows a 3-D drawing of an exemplary blade protection device latched in a guard-retracted mode.

FIG. 1B shows a 3-D drawing of an exemplary blade protection device latched in a guard-extended mode.

In a preferred embodiment, the present invention consist essentially of two parts. An instrument body 12 that is preferably solid and has a reduced diameter blade-carrying end 16 and a slidably mountable guard 14 that may, for instance, be a sliding hollow cylindrical guard that surrounds the instrument body 12 and that may be locked into place to either cover the blade in a guard-extended position, or be secured away from the blade during blade use in a guard-retracted position.

In a preferred embodiment, the instrument body 12 is further characterized by a protrusion 22 located proximate to the blade-carrying end 16. The protrusion 22 serves to secure the guard in place when covering the blade in the guard-extended position.

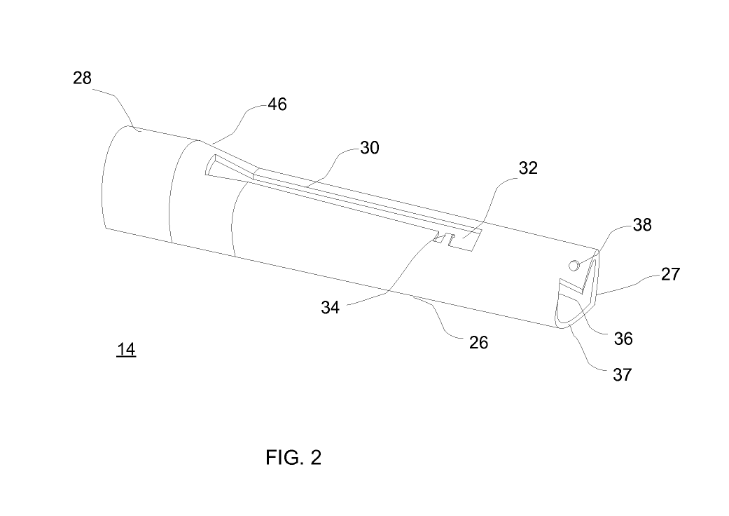

FIG. 2 shows a 3-D drawing of an exemplary slidably mountable guard 14.

In a preferred embodiment, the slidably mountable guard 14 is characterized by a flared opening at the blade 20 covering end, and includes a channel 30 that is not continuous with the flared opening, but is set back for the opening end, and into which the protrusion 22 fits. The channel 30 may end in an L-shaped extension 32. To lock the guard in the guard-extended, or closed, position, the slidably mountable guard 14 is slid down over the blade 20, which repositions the protrusion 22 up toward the L-shaped extension 32. The instrument body 12 is then twisted relative to the slidably mountable guard 14 to insert the protrusion 22 into the L-shaped extension 32.

The slidably mountable guard 14 may include a “V” notch that leads into a shord detent cut in the guard body. A metal pin located proximate to the distal end 18 of the instrument body 12 may slide into the “V” notch and be forced by twisting into the detaining cut 38.

In a further preferred embodiment, the distal end 27 of the slidably mountable guard 14 is not defined by a uniform plane, but by an angled slice 37. The angled slice 37 leads into a shallow, L-shaped locating notch 36. The locating notch 36 acts as a guide to a locating element 40 affixed to the instrument body 12 to the detaining cut 38.

The exemplary slidably mountable guard 14 shown in FIG. 2 further includes a substantially conical section 46 that connects the guard section 28 to the tubular section 26, and in which the channel 30 extends partway into the substantially conical section 46 thereby enabling the protrusion 22 to slide into the channel 30.

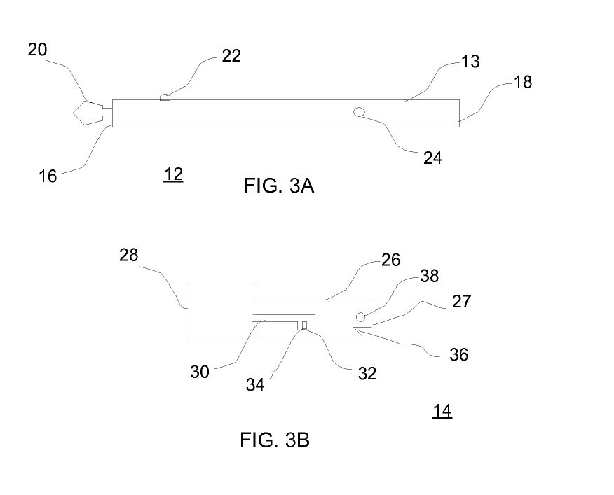

FIG. 3A shows a further embodiment of the instrument body 12 of a blade protection device 10 of the present invention. The instrument body 12 may have a blade 20 attached at a blade-carrying end 16, a protrusion 22 located proximate to the blade-carrying end 16 and an indentation 24 located proximate to a distal end 18. The protrusion 22 may for instance be, but is not limited to, a solid protrusion, a lug or a tab. The indentation 24 may be adapted to accept a locating element 40. The locating element 40 may for instance be, but is not limited to, a metal pin, a metal ball-bearing, a non-metallic pin or a non-metallic sphere. The blade 20 may for instance be, but is not limited to, a surgical scalpel blade.

FIG. 3B shows a further embodiment of the slidably mountable guard 14 of a blade protection device 10 of the present invention. The slidably mountable guard 14 may have a tubular section 26 that may be joined to a guard section 28. The tubular section 26 may include a channel 30 that may for instance, extend from a junction of the tubular section 26 and the guard section 28 to a predetermined position along the tubular section 26 related to a guard-extended latch 42.

The guard-extended latch 42 may for instance, operate using an L-shaped extension 32 of the channel 30 and the protrusion 22, as described later in more detail.

The tubular section 26 is preferably sized to slidebly fit the instrument body 12.

The slidably mountable guard 14 may include part of a guard-retracted latch 44 such as, but not limited to, a locating notch 36 and a detaining cut 38.

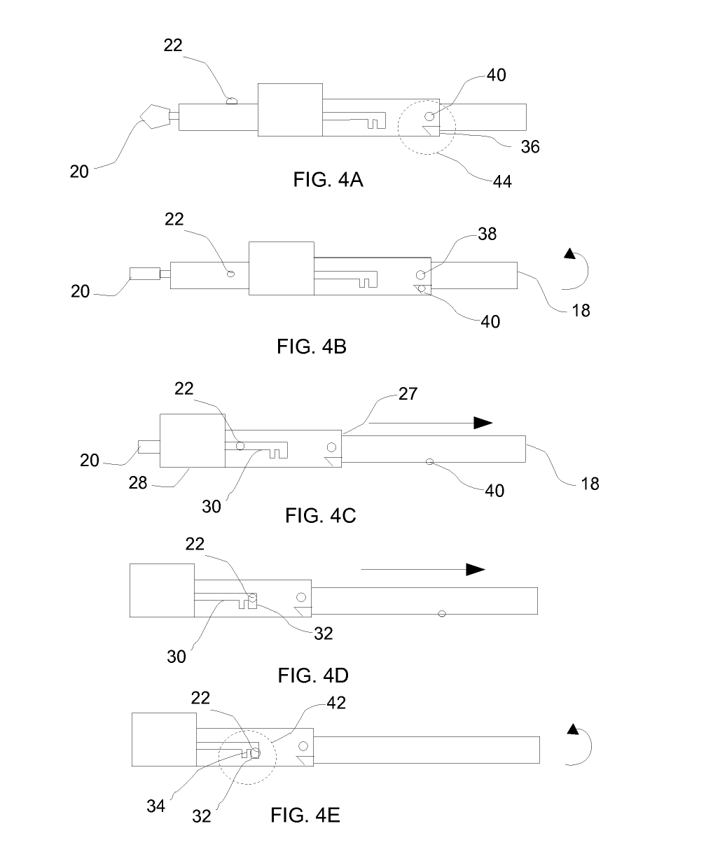

FIG. 4A shows an elevation view of an exemplary blade protection device 10 latched in a guard-retracted position, with the exposed blade 20 seen in plan elevation. The guard-refracted latch 44 may for instance, be effected by a combination of the locating notch 36, the detaining cut 38 and a locating element 40 mounted in the indentation 24 of the instrument body 12. To engage the extended-position latch 44, the slidably mountable guard 14 may be slid back along the instrument body 12 with the protrusion 22 sliding along the channel 30 until the locating notch 36 engages with the locating element 40 attached to the instrument body 12. The slidably mountable guard 14 may then be twisted anti-clockwise, forcing the locating element 40 under the slightly flexible body of the tubular section 26 until it reaches the detaining cut 38 and engages, latching the blade protection device 10 in a guard-retracted mode with the blade 20 exposed for use.

FIG. 4B shows an elevation view of an exemplary blade protection device unlatched in a guard-retracted position, with the blade 20 now seen in side elevation. The blade protection device 10 has been unlatched by twisting or rotating the instrument body 12 relative to the slidably mountable guard 14. The rotation has forced the locating element 40 out of the detaining cut 38 and under the slightly flexible tubular section 26 to the locating notch 36.

FIG. 4C shows an elevation view of an exemplary blade protection device transitioning from a guard-retracted position to a guard-extended position, with the blade 20 seen in side elevation and now partially covered by the guard section 28 of the slidably mountable guard 14. As the distal end 18 of the instrument body 12 becomes more separated from the distal end 27 of the slidably mountable guard 14, the protrusion 22 on the instrument body 12 slides along the channel 30 in the tubular section 26.

FIG. 4D shows an elevation view of an exemplary blade protection device unlatched in a guard-extended position, with the blade 20 now completely inside the guard section 28. The protrusion 22 has reached a limit of travel imposed by the guard-extended latch 42. This may, for instance be, an L-shaped extension 32 that prevents the slidably mountable guard 14 sliding further towards the proximate or blade-carrying end 16 of the instrument body 12.

FIG. 4E shows an elevation view of an exemplary blade protection device latched in a guard-extended position, with the blade 20 now completely inside the guard section 28 and the instrument body 12 latched to the slidably mountable guard 14 so that the slidably mountable guard 14 can no longer slide in either direction along the instrument body 12.

The guard-extended latch 42 may for instance, be effected by rotating the instrument body 12 with respect to the slidably mountable guard 14 so that the protrusion 22 is forced down the L-shaped extension 32 and latched in place by the retaining lug 34. To unlatch the blade protection device 10, the instrument body 12 may be rotated in the opposite direction with respect to the slidably mountable guard 14 so that the protrusion 22 aligns with the channel 30 and the slidably mountable guard 14 may be withdrawn to expose the blade 20.

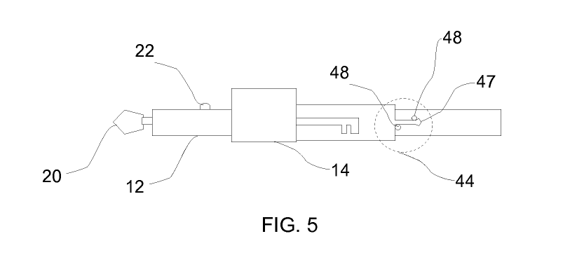

FIG. 5 shows an elevation view of a further exemplary blade protection device latched in a guard-retracted position.

In this embodiment, there is a resiliently biased tab 47 extending from the distal end 27 of the slidably mountable guard 14, and two counter-biased retaining pins 48 attached to the instrument body 12. To form the guard-retracted latch 44, the resiliently biased tab 47 engages with the counter-biased retaining pins 48.

Although every reasonable attempt is made in the accompanying drawings to represent the various elements of the embodiments in relative scale, it is not always possible to do so with the limitations of two-dimensional paper. Accordingly, in order to properly represent the relationships of various features among each other in the depicted embodiments and to properly demonstrate the invention in a reasonably simplified fashion, it is necessary at times to deviate from absolute scale in the attached drawings. However, one of ordinary skill in the art would fully appreciate and acknowledge any such scale deviations as not limiting the enablement of the disclosed embodiments.

Although the invention has been described in language specific to structural features and/or methodological acts, it is to be understood that the invention defined in the appended claims is not necessarily limited to the specific features or acts described. Rather, the specific features and acts are disclosed as exemplary forms of implementing the claimed invention. Modifications may readily be devised by those ordinarily skilled in the art without departing from the spirit or scope of the present invention.

1. A blade protection device, comprising:
an instrument body having a blade-carrying end;
a slidably mountable guard comprising:
a tubular section adapted to slidably fit said instrument body;
a guard section, joined to said tubular section, and shaped to enclose said blade in a guard-extended position;
an elongated hole in said mountable guard, extending substantially from the junction of said tubular and guard sections to a predetermined position along said tubular section and shaped to slidably accommodate a protrusion on said instrument body; and
an L-shaped extension to said elongated hole, shaped to releasably engage said protrusion thereby providing a guard-extended latch for latching said blade within said guard section; and
a guard-retracted latch for latching said blade so as to protrude from said guard section.

2. The device of claim 1 wherein said tubular section further comprises a locating notch and a detaining cut proximate to the distal end of said tubular section; said instrument body further comprises an indent shaped to accommodate a locating element proximate to the distal end; and wherein said locating notch, said detaining cut and said locating element operate to provide said guard-retracted latch.

3. The device of claim 1 wherein said slidably mountable guard further comprises a flared section that joins said tubular section to said guard section.

4. The device of claim 1 wherein said tubular section further comprises a tab extending from the distal end of said tubular section; said instrument body further comprises two counter biased pins; and wherein said tab and said counter biased pins operate to provide said guard-retracted latch.

5. A blade protection method, comprising:
providing an instrument body having a blade-carrying end;
slidably mounting a slidable guard on said instrument body, said slidable guard comprising:
a tubular section adapted to slidably fit said instrument body;
a guard section, joined to said tubular section, and shaped to enclose said blade in a guard-extended position;
an elongated hole in said mountable guard, extending substantially from the junction of said tubular and guard sections to a predetermined position along said tubular section and shaped to slidably accommodate a protrusion on said instrument body; and
an L-shaped extension to said elongated hole, shaped to releasably engage said protrusion thereby providing a guard-extended latch for latching said blade within said guard section; and
providing a guard-retracted latch for latching said blade so as to protrude from said guard section.

6. The method of claim 5 comprising further providing a locating notch and a detaining cut in said tubular section, proximate to the distal end of said tubular section; providing an indent on said instrument body, said indent shaped to accommodate a locating element proximate to the distal end of said instrument body; and wherein said locating notch, said detaining cut and said locating element operate to provide said guard-retracted latch.

7. The method of claim 5 further comprising providing a flared section that joins said tubular section of said slidebly mountable guard to said guard section.

8. The method of claim 5 further comprising providing a tab extending from the distal end of said tubular section; further comprising providing two counter biased pins said instrument body; and wherein said tab and said counter biased pins operate to provide said guard-refracted latch.
